(19) United States
(12) Patent Application Publication
(10) Pub. No.: US 2010/0158925 A1
(43) Pub. Date: Jun. 24, 2010

(54) Lyophilized formulations of anti-egfr antibodies

(76) Inventors: Meera Agarkhed, Lawrenceville, NJ (US); Arvind Srivastava, Stewartsville, NJ (US); Joel Goldstein, Flemington, NJ (US)

Correspondence Address: IMCLON;Lerner, David, Littenberg, Krumholz & Mentlik, LLP, 600 South Avenue West, Westfield, NJ, 07090, US

(21) Appl. No.: 12/308,451

(22) PCT Filed: Jun. 13, 2007
(86) PCT No.: PCT/US2007/071119

§ 371 Date: Dec. 12, 2008

(51) Int. Cl. A61K 39/395

(52) U.S. Cl. 424/172.1

(57) ABSTRACT

In one embodiment, the present invention provides a stable lyophilized formulation comprising an anti-EGFR antibody, preferably cetuximab; lactobionic acid; and a buffer, preferably histidine. In one preferred embodiment, the present invention provides a stable lyophilized formulation comprising about 50 mg/mL to about 140 mg/mL of ERBITUX?, about 0.125% lactobionic acid, about 25 mM histidine buffer at a pH of about 6.0, about 0.005% Tween 80, and about 1.875% glycine.

### CROSS-REFERENCE TO RELATED APPLICATION

This application claims the benefit of priority from U.S. Provisional Patent Application Ser. No. 60/813,958 filed Jun. 14, 2006, the contents of which are incorporated herein by reference.

### FIELD OF INVENTION

The present invention relates to formulations and methods useful for the stabilization of antibodies that bind to epidermal growth factor receptor (EGFR) antibodies. More particularly, this invention relates to the formulation of anti-EGFR antibodies, especially cetuximab, with lactobionic acid in a histidine buffer.

### BACKGROUND OF THE INVENTION

To realize the clinical potential of antibodies, their biological activity must be retained during storage and administration. Both chemical and physical instability can contribute to a decrease in biological activity. The antibodies may undergo aggregation, oxidation, deamidation, or hydrolysis due to water and temperature fluctuations. One way to retain the biological activity of antibodies is to stabilize an antibody formulation by lyophilization. Particularly useful lyophilized formulations provide a high antibody concentration upon reconstitution. There is a need for stable lyophilized formulations of anti-EGFR antibodies.

### BRIEF SUMMARY OF THE INVENTION

Accordingly, the present invention provides a stable aqueous formulation suitable for lyophilization comprising an anti-EGFR antibody in a protein concentration ranging from about 50 mg/mL to about 140 mg/mL, lactobioinic acid, and a buffer. The anti-EGFR antibody is preferably cetuximab. The lactobionic acid is present in a concentration of from about 0.1% to about 0.5%, or more preferably in a concentration of from about 0.125% to about 0.25%.

The formulation is preferably buffered to a pH of about 6.0, the buffer is preferably present in a concentration of about 25 mM, and the buffer is preferably a histidine buffer.

The stable aqueous formulation further includes stabilizing agents selected from the group consisting of mannitol, glycine and combinations thereof, as well as a surfactant. The surfactant is preferably polyoxyethylene(20)sorbitan monooleate, polyoxyethylene-polyoxypropylene block copolymer, and/or a combination thereof.

The invention also provides for a lyophilized anti-EGFR formulation prepared by freezing and drying the inventive stable aqueous formulation described supra.

The invention further provides a method of stabilizing an anti-EGFR antibody comprising formulating the antibody with the inventive stable aqueous formulation described supra.

In addition, the invention provides a method of treating a mammal, e.g., a human, comprising administering a therapeutically effective amount of a reconstituted lyophilized formulation of as described supra, to a mammal in need thereof.

In summary, the present invention provides a stable lyophilized formulation comprising an anti-EGFR antibody, preferably cetuximab; lactobionic acid; and a buffer, preferably histidine. In preferred embodiments, the protein concentration is about 50 mg/mL to about 140 mg/mL. In addition to the lactobionic acid, the formulation may contain one or more stabilizing agents such as mannitol and glycine. The formulation may also contain a surfactant such as Tween 80® (polyoxyethylene(20)sorbitan monooleate) or Pluronic F-68® (Polyoxyethylene-polyoxypropylene block copolymer). In one preferred embodiment, the present invention provides a stable lyophilized formulation comprising about 50 mg/mL to about 140 mg/mL of ERBITUX®, about 0.125% lactobionic acid, about 25 mM histidine buffer at a pH of about 6.0, about 0.005% Tween 80, and about 1.875% glycine.

The present invention also provides a method of stabilizing an antibody comprising lyophilizing an aqueous formulation comprising an anti-EGFR antibody, preferably cetuximab; lactobionic acid; and a buffer, preferably histidine.

The present invention also provides methods of treatment comprising: administering a reconstituted formulation to a mammal, such as a human in need thereof. In the case of treatment with cetuximab, the amount administered is commensurate with the amounts known to those of ordinary skill.

### BRIEF DESCRIPTION OF THE FIGURES

FIG. 1 shows turbidity as a function of time for cetuximab in the presence and absence of Tween 80®.

FIG. 2 shows the turbidity of cetuximab solutions in various formulation conditions after 72 hours of incubation at 50° C.

FIG. 3 shows the percentage material loss of cetuximab in various formulation conditions after 72 hours of incubation at 50° C.

FIG. 4 shows the monomer percentage of cetuximab in various formulation conditions after 72 hours of incubation at 50° C.

FIG. 5 shows the percentage of soluble aggregates of cetuximab in various formulation conditions after 72 hours of incubation at 50° C.

FIG. 6 shows the percentage of degradants of cetuximab in various formulation conditions after 72 hours of incubation at 50° C.

FIG. 7 shows the turbidity of various reconstituted cetuximab lyophilized products for varying incubation times.

FIG. 8 shows the monomer percentage of various reconstituted cetuximab lyophilized products for varying incubation times.

FIG. 9 shows the percentage of soluble aggregate of various reconstituted MAb cetuximab lyophilized products for varying incubation times.

FIG. 10 shows the percentage of degradants of various reconstituted cetuximab lyophilized products for varying incubation times.

### DETAILED DESCRIPTION OF THE INVENTION

The present invention provides stable lyophilized formulations comprising an anti-EGFR antibody, lactobionic acid, and a buffer. The formulations may comprise additional elements such as stabilizing agents, surfactants, reducing agents, carriers, preservatives, amino acids, and chelating agents. The present invention also provides methods of stabilization comprising lyophilizing an aqueous formulation of an anti-EGFR antibody. The formulations can be lyophilized to stabilize the anti-EGFR antibodies during processing and storage, and then the formulations can be reconstituted for pharmaceutical administration. Preferably, the antibody essentially retains its physical and chemical stability and integrity from production to administration. Various formulation components maybe suitable to enhance stability according to the present invention, including buffers, pH, surfactants, sugars, sugar alcohols, sugar derivatives, and amino acids.

The formulations of the present invention are lyophilized. Lyophilization is a stabilizing process in which a substance is first frozen and then the quantity of the solvent is reduced, first by sublimation (the primary drying process) and then desorption (the secondary drying process) to values that will no longer support biological activity or chemical reactions, in a lyophilized formulation, the hydrolysis, deamidation, and oxidation reactions associated with solutions can be avoided or slowed significantly. A lyophilized formulation may also avoid damage due to short-term temperature excursions during shipping. The formulations of the present invention may also be dried by other methods known in the art such as spray drying and bubble drying. Unless otherwise specified, the formulations of the present invention are described in terms of their component concentrations as measured in the formulation before lyophilization.

The formulations of the present invention contain lactobionic acid as a stabilizing agent. The lactobionic acid concentration is preferably about 0.1% to about 0.5%, more preferably about 0.125% to about 0.25%, and most preferably about 0.125%, (weight/volume).

In preferred embodiments, the lyophilized formulation provides a high concentration of the anti-EGFR antibody upon reconstitiition. Preferably, the antibody concentration in the aqueous formulation before lyophilization is about 10 mg/mL to about 140 mg/mL, more preferably about 50 to about 140 mg/mL, and most preferably about 50 mg/mL. In preferred embodiments, the stable lyophilized formulation is reconstitutable with a liquid to form a solution with an antibody concentration about 1-10 times higher than the antibody concentration of the formulation before lyophilization. For instance, in one embodiment, the lyophilized formulation is reconstituted with 1 mL milliQ water or less to obtain a particle-free reconstituted formulation with an antibody concentration of about 50 mg/mL to about 200 mg/mL.

Various analytical techniques known in the art can measure the antibody stability of a reconstituted lyophilized formulation. Such techniques include, for example, determining (i) thermal stability using differential scanning calorimetry (DSC) to determine the main melting temperature (Tm); (ii) mechanical stability using controlled agitation at room temperature; (iii) real-time isothermal accelerated temperature stability at temperatures of about −20° C., about 4° C., room temperature (about 23° C.-27° C.), about 40° C., and about 50° C; (iv) solution turbidities by monitoring absorbance at about 350 nm and (v) the amount of monomer, aggregates and degradants using SEC-HPLC (size exclusion chromatography-high performance liquid chromatography). Stability can be measured at a selected temperature for a selected time period. In a preferred embodiment, the formulation is stable at 60° C. for at least about 96 hours and at room temperature for at least 1 month.

The antibodies of the present invention can be monoclonal or polyclonal antibodies or any other suitable type of an antibody, such as a fragment or a derivative of an antibody, a single chain-antibody (scFv), or a synthetic homologue of the antibody, provided that the antibody has the same binding characteristics as, or that have binding characteristics comparable to, those of the whole antibody. As used herein, unless otherwise indicated or clear from the context, antibody domains, regions and fragments are accorded standard definitions as are well known in the art. See, e.g., Abbas et al., Cellular and Molecular Immunology, W. B. Saunders Company, Philadelphia, Pa. (1991).

Cleaving a whole antibody can produce antibody fragments. Antibody fragments can also be produced by expressing DNA that encodes the fragment. Fragments of antibodies can be prepared by methods described by Lamoyi et al., J. Immunol. Methods, 56:235-243 (1983) and by Parham, J. Immunol. 131: 2895-2902 (1983). Such fragments can contain one or both Fab fragments or the F(ab′)₂ fragment. Such fragments can also contain single-chain fragment variable region antibodies, i.e. scFv, diabodies, or other antibody fragments. Preferably the antibody fragments contain all six complementarity-determining regions of the whole antibody, although fragments containing fewer than all of such regions, such as three, four or five CDRs, can also be functional. The antibody fragment can also be conjugated to a carrier molecule. Some suitable carrier molecules include keyhole limpet hemocyanin and bovine serum albumen. Conjugation can be carried out by methods known in the art.

Antibodies of the present invention also include those for which binding characteristics have been improved by direct mutation, methods of affinity maturation, phage display, or chain shuffling. Affinity and specificity can be modified or improved by mutating CDRs arid screening for antigen binding sites having the desired characteristics (see, e.g., Yang et al., J. Mol. Bio., 254: 392-403 (1995)). CDRs are mutated in a variety of ways. One way is to randomize individual residues or combinations of residues so that in a population of otherwise identical antigen binding sites, all twenty amino acids are found at particular positions. Alternatively, mutations are induced over a range of CDR residues by error prone PCR methods (see, e.g., Hawkins et al., J. Mol. Bio., 226: 889-896 (1992)). Phage display vectors containing heavy and light chain variable region genes are propagated in mutator strains of E. coli (see, e.g., Low et al., J. Mol. Bio. 250: 359-368 (1996)). These methods of mutagenesis are illustrative of the many methods known to one of skill in the art.

The antibodies of the present invention can also be bispecific and/or multivalent. A variety of chemical and recombinant methods have been developed for the production of bispecific and/or multivalent antibody fragments. For a review, see Holliger and Winter, Curr. Opin. Biotechnol. 4:446-449 (1993); Carter et al., J. Hematotherapy 4:462-470 (1995); Plückthun and Pack, Immunotechnology 3, 83-105 (1997). Bispecificity and/or bivalency has been accomplished by fusing two scFv molecules via flexible linkers, leucine zipper motifs, C_{H}C_{L}-heterodimerization, and by association of scFv molecules to form bivalent monospecific diabodies and related structures. The addition of multimerization sequences at the carboxy or amino terminus of the scFv or Fab fragments has achieved multiyalency, by using, for example, p53, streptavidin, and helix-turn-helix motifs. For example, by dimerization via the helix-turn-helix motif of an scFv fusion protein of the form (scFv1)-hinge-helix-turn-helix-(scFv2), a tetravalent bispecific miniantibody is produced having two scFv binding sites for each of two target antigens. Improved avidity can also been obtained by providing three functional antigen binding sites. For example, scFv molecules with shortened linkers connecting the V_{H} and V_{L} domains associate to form a triabody (Kortt et al., Protein Eng. 10:423-433 (1997)).

Production of IgG-type bispecific antibodies, which resemble IgG antibodies in that they possess a more or less complete IgG constant domain structure, has been achieved by chemical cross-linking of two different IgG molecules or by co-expression of two antibodies from the same cell. One strategy developed to overcome unwanted pairings between two different sets of IgG heavy and light chains co-expressed in transfected cells is modification of the C_{H}3 domains of two heavy chains to reduce homodimerization between like antibody heavy chains. Merchant et al., Nat. Biotechnology 16: 677-681 (1998). In that method, light chain mispairing was eliminated by requiring the use of identical light chains for each binding site of those bispecific antibodies.

In some cases, it is desirable to maintain functional or structural aspects other than antigen specificity. For example, both complement-mediated cytotoxicity (CMC) and antibody-dependent cell-mediated cytotoxicity (ADCC), which require the presence and function of Fc region heavy chain constant domains, are lost in most bispecific antibodies. Coloma and Morrison created a homogeneous population of bivalent BsAb molecules with an Fc domain by fusing a scFv to the C-terminus of a complete heavy chain. Co-expression of the fusion with an antibody light chain resulted in the production of a homogeneous population of bivalent, bispecific molecules that bind to one antigen at one end and to a second antigen at the other end (Coloma and Morrison, Nat. Biotechnology 15, 159-163 (1997)). However, this molecule had a reduced ability to activate complement and was incapable of effecting CMC. Furthermore, the C_{H}3 domain bound to high affinity Fc receptor (FcγR1) with reduced affinity. Zhu et al., PCT/US01/16924, have described the replacement of Ig variable domains with single chain Fvs in order to produce tetrameric Ig-like proteins that (1) are bispecific and bivalent, (2) are substantially, homogeneous with no constraints regarding selection of antigen-binding sites, (3) comprise Fc constant domains and retain associated functions, and (4) can be produced in mammalian or other cells without further processing. By a similar method, bispecific monovalent Fab-like proteins or polypeptides can be produced.

Preferably, the antibodies of the present invention are monoclonal antibodies. The antibodies of the present invention can be chimeric antibodies having a variable region of an antibody of one species, for example, a mouse, and a constant region of an antibody of a different species, for example, a human. Alternatively, the antibodies of the present invention can be humanized antibodies having hypervariable or complementarity-determining regions (CDRs) of an antibody from one species, for example, a mouse, and framework variable regions and a constant region of a human antibody. Also alternatively, the antibodies of the present invention can be human antibodies having both a constant region and a variable region of a human antibody.

As used herein, “antibodies” and “antibody fragments” include modifications that retain specificity for the EGF receptor. Such modifications include, but are not limited to, conjugation to an effector molecule such as a chemotherapeutic agent (e.g., cisplatin, taxol, doxorubicin) or cytotoxin (e.g., a protein or a non-protein organic chemotherapeutic agent). The antibodies can be modified by conjugation to detectable reporter moieties. Also included are antibodies with alterations that affect non-binding characteristics such as half-life (e.g., pegylation).

Equivalents of the antibodies, or fragments thereof, of the present invention also include polypeptides with amino acid sequences substantially the same as the amino acid sequence of the variable or hypervariable regions of the full-length anti-EGFR antibodies. Substantially the same amino acid sequence is defined herein as a sequence with at least 70%, preferably at least about 80%, and more preferably at least about 90% homology to another amino acid sequence, as determined by the FASTA search method in accordance with Pearson and Lipman (Proc. Natl. Acad. Sci. USA 85, 2444-8 (1988)).

In a preferred embodiment, the anti-EGFR antibody binds EGFR and blocks binding of a ligand, such as EGF or TNF-a, to EGFR. This blockage results in inhibition of tumor growth, which includes inhibition of tumor invasion, metastasis, cell repair, and angiogenesis, by interfering with the effects of EGFR activation. Accordingly, a preferred anti-EGFR antibody is cetuximab. Cetuximab is a chimeric antibody (trademarked as ERBITUX® and also known as C225) which has a molecular weight of about 152 kDa and an isoelectric point of about 8.0. Cetuximab binds EGFR and blocks binding of a ligand. In addition, or alternatively, cetuximab may promote internalization of the receptor-antibody complex, preventing further stimulation of the receptor by its ligand or any other mechanism. Further characterization of cetuximab is disclosed in U.S. application Ser. No. 08/973,065 (Goldstein et al.) and Ser. No. 09/635,974 (Teufel); WO 99/60023 (Waksal et al) and WO 00/69459 (Waksal), all of which are incorporated by reference herein.

The amino acid sequences of the chimeric heavy and light chains of cetuximab are provided by SEQ ID NOS:2 and 4, respectively. SEQ ID NOS:1 and 3 provide the respective nucleotide sequences encoding the chimeric antibody chains. In an embodiment of the invention, the antibody heavy and light chains arc expressed with cleavable signal sequences that direct transport and secretion in a host cell. SEQ ID NOS:6 and 8 respectively provide amino acid sequences of amino terminal signal peptides of cetuximab heavy and light chains. SEQ ID NOS:5 and 7 provide the encoding nucleotide sequences.

In another embodiment, the anti-EGFR antibody is a fully human, monoclonal antibody specific for EGFR, such as, for example, panitumumab (formerly ABX-EGF; Abgenix, Inc). ABX-EFG binds EGFR with high specificity, blocking binding of EGFR to both its ligands, EGF and TNF-alpha. The sequence and characterization of ABX-EGF is disclosed in U.S. Pat. No. 6,235,883 at col. 28, line 62 through col. 29, line 36 and in FIG. 29-34, which is incorporated by reference herein. See also Yang et al., Critical Rev. Oncol./Hematol., 38 (1): 7-23, 2001, which is also incorporated by reference herein.

In another embodiment, the anti-EGFR antibody is a humanized monoclonal antibody specific for EGFR, such as, for example, matuzumab (formerly EMD 72000; Merck KGaA). Matuzumab binds EGFR with high specificity and inhibits ligand binding. The sequence and characterization of matuzumab is disclosed in U.S. Pat. No. 5,558,864 at col. 19, line 43 through col. 20, line 67, which is incorporated by reference herein. See also, Kettleborough et al, Prot. Eng., 4 (7): 773-83, 1991. Nimotuzumab (TheraCIM h-R3; YM Biosciences, Inc.) is another example of a humanized antibody. The sequence and characterization of nimotuzumab is disclosed in U.S. Pat. No. 5,891,996 at col. 10, line 54 through col. 13, line 6. See also Mateo at al., Immunotechnology, 3; 71-81, 1997.

According to the present invention, a buffer may be used to maintain the pH of the formulation. The buffer minimizes fluctuations in pH due to external variations. The formulations of the present invention contain one or more buffers to provide the formulations at a suitable pH, preferably about 5.5 to about 6.5, and most preferably about 6.0. Exemplary buffers include, but are not limited to organic buffers generally, such as histidine, malate, tartrate, succinate, and acetate. Preferably, the buffer is histidine. The buffer concentration is preferably about 5 mM to about 50 mM, more preferably about 10 mM to about 25 mM, and most preferably about 25 mM. A particularly preferred buffer is about 25 mM histidine at a pH of about 6.0.

The formulations of the present invention may contain one or more surfactants. Antibody solutions have high surface tension at the air-water interface. In order to reduce this surface tension, antibodies tend to aggregate at the air-water interface. A surfactant minimizes antibody aggregation at the air-water interface, thereby helping to maintain the biological activity of the antibody in solution. For example, adding 0.01% Tween 80® can reduce antibody aggregation in solution. When the formulation is lyophilized, the surfactant may reduce the formation of particulates in the reconstituted formulation. In the lyophilized formulations of the present invention, the surfactant can be added to one or more of the pre-lyophilized formulation, the lyophilized formulation, and the reconstituted formulation, but preferably the pre-lyophilized formulation. For example, 0.005% Tween 80® can be added to the antibody solution before lyophilization. Surfactants include, but are not limited to Tween 20® (Polyoxyethylene-20-Sorbitan Monolaurate), Tween 80®, Pluronic F-68®, and bile salts. Tween 80® and Pluronic F-68 are preferred. The surfactant concentration is preferably about 0.001% to about 0.01%, more preferably about 0.005% to about 0.01%, and most preferably about 0.005%, (weight/volume). Most preferably, the surfactant is about 0.005% Tween 80®.

The formulations of the present invention may contain one or more stabilizing agents in addition to the lactobionic acid. A stabilizing agent helps to prevent aggregation and degradation of the antibodies. Suitable stabilizing agents include, but are not limited to polyhydric sugars, sugar alcohols, sugar derivatives, and amino acids. Preferred stabilizing agents include, but are not limited to glycine, trehalose, mannitol, and sucrose. In a preferred embodiment, the additional stabilizing agent is glycine, or both glycine and mannitol. The concentration of each additional stabilizing agent is preferably about 0.1% to about 2%, more preferably about 1% to about 2%, and most preferably about 2%. A particularly preferred additional stabilizing agent is 1.875% glycine.

Stabilizing agents and surfactants may be used alone or in combination with one another to help stabilize the antibody solution. In one preferred embodiment, the present invention provides a stable lyophilized formulation comprising about 50 mg/mL to about 140 mg/mL of ERBITUX®, about 0.125% lactobionic acid, about 25 mM histidine buffer at a pH of about 6, about 0.005% Tween 80®, and about 1.875% glycine.

The lypohilization process can generate a variety of stresses that may denature proteins or polypeptides. These stresses include temperature decrease, ice crystal formation, ionic strength increase, pH changes, phase separation, removal of hydration shell, and concentration changes. Antibodies that are sensitive to the stresses of the freezing and/or drying process can be stabilized by adding one or more cryo- and/or lyoprotectants. A cryo- or lyoprotectant may be, for example, a sugar such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and combinations thereof. Examples of preferred lyoprotectants include, but are not limited to the stabilizing agents and surfactants as described above.

The present invention also provides a method of treatment comprising administering a reconstituted formulation. The reconstituted formulations are prepared by reconstituting the lyophilized formulations of the present invention, for example with 1 mL milliQ water. The reconstitution time is preferably less than 1 minute. As described above with regard to antibody concentration, in particular embodiments, the stable lyophilized formulation is reconstitutable with a liquid to form a concentrated reconstituted formulation with an antibody concentration about 1-10 times higher than the antibody concentration of the formulation before lyophilization. The concentrated reconstituted formulation allows for flexibility in administration. For example, the reconstituted formulation can be administered in a dilute form intravenously or it can be administered in a more concentrated form by injection. A concentrated reconstituted formulation of the present invention can be diluted to a concentration that is tailored to the particular subject and/or the particular route of administration. Accordingly, the present invention provides methods of treatment comprising administering a therapeutically effective amount of an anti-EGFR antibody to a mammal, particularly a human, in need thereof. The term administering as used herein means delivering the antibodies of the present invention to a mammal by any method that can achieve the result sought. They can be administered, for example, intravenously or intramuscularly. In one embodiment, a concentrated reconstituted formulation is administered by injection.

Therapeutically effective amount means an amount of antibody of the present invention that, when administered to a mammal, is effective in producing the desired therapeutic effect, such as neutralizing EGFR activity, inhibition of tumor growth, or treating a non-cancerous hyperproliferative disease. Administration of the antibodies as described above can be combined with administration of other antibodies or any conventional treatment agent, such as an anti-neoplastic agent.

The present invention is further described by the following non-limiting examples. Any cited references are incorporated herein by reference, including Sambrook J, Fritsch E F, Maniatis T. Molecular Cloning: A Laboratory Manual. Plainview, N.Y.: Cold Spring Harbor Laboratory Press; 1989.

### Examples

### Example 1

### Aggregation Study

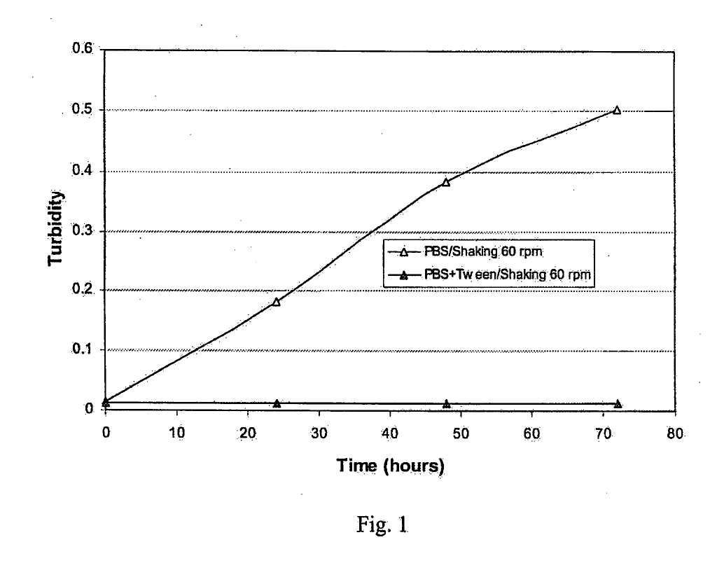

The stability of Cetuximab for eventual lyophilization was considered. A solution of cetuximab (5 mg/mL) in phosphate-buffered saline (PBS) and a solution of cetuximab (5 mg/mL) in PBS containing 0.01% Tween 80® were prepared. Each solution (3 ml) was rocked at 60 rpm at 4° C. Solution turbidity was measured at 540 nm. The results are shown in FIG. 1 as a graph plotting turbidity of the each solution versus time. In the absence of Tween 80®, turbidity increased with time. In the presence of Tween 80® (0.01%), the turbidity remained unchanged. Thus, 0.01% Tween 80® minimized the aggregation of cetuximab at the air-water interface.

### Example 2

### Real Time Solution Stability

The real time stability of cetuximab in solution was measured by varying the buffers and excipients. Various solutions of cetuximab (2 mg/mL) were prepared at pH 6.0 using each of the following buffers (25 mM):

(i) malate,

(ii) histidine,

(iii) tartrate,

(iv) succinate, and

(v) acetate.

For each buffer, a solution with the following excipient(s) was prepared:

(i) 0.01% ®80;

(ii) 0.01% Tween 80® and 0.25% lactobionic acid;

(iii) 0.01% Tween 80®, 0.25% lactobionic acid, and 2% glycine;

(iv) 0.01% Tween 80® and 2% sucrose;

(v) 0.01% Tween 80®, 2% sucrose, and 2% glycine;

(vi) 0.01% Tween 80® and 2% trehalose;

(vii) 0.01% Tween 80®, 2% trehalose, and 2% glycine.

The various solutions were incubated at 50° C. for 72 hours.

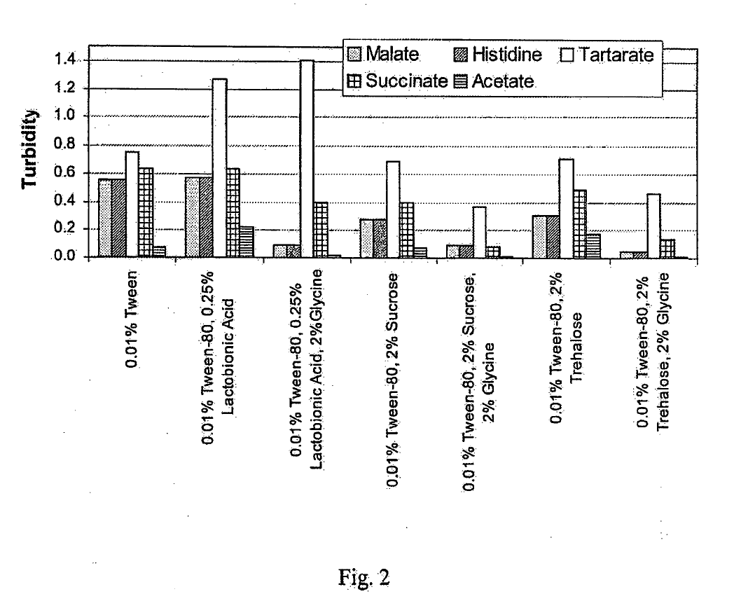

Turbidity was measured at 540 nm. FIG. 2 shows the turbidities under various formulation conditions after incubation at 50° C. for 72 hours. The solution turbidity was least in histidine buffer and highest in tartrate buffer. The excipient combination of 0.01% Tween 80®, 2% sugar (sucrose or trehalose), and 2% glycine reduced the turbidity in all buffers. The 0.25% lactobionic acid alone did not have much effect, but with 2% glycine, it reduced the solution turbidity in all buffers.

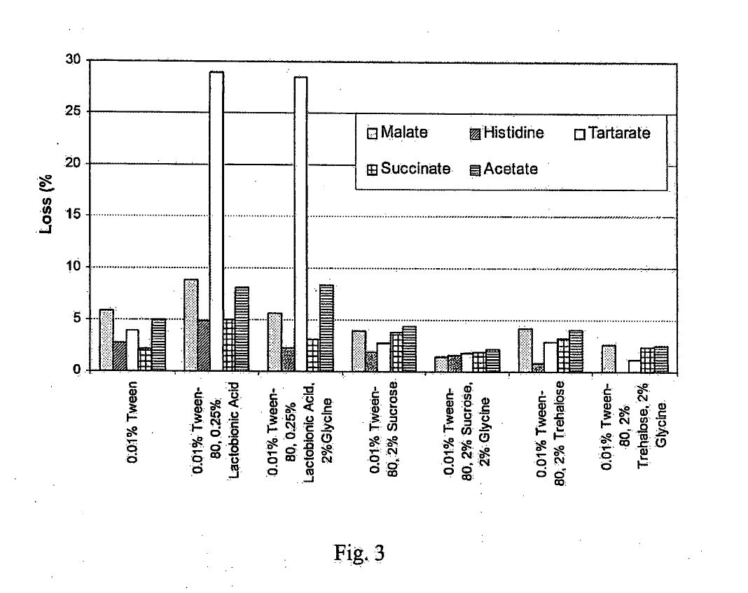

SEC-HPLC analysis was used to measure monomer, aggregate and degradant fraction content. The percentage material loss was estimated by calculating the differences in the total peak area between the initial samples and the samples after incubation at 50° C for 72 hours. FIG. 3 shows the percentage of material loss under the various formulations conditions after incubation at 50° C for 72 hours. The percentage of material lost was the largest in tartrate buffer and smallest in histidine buffer.

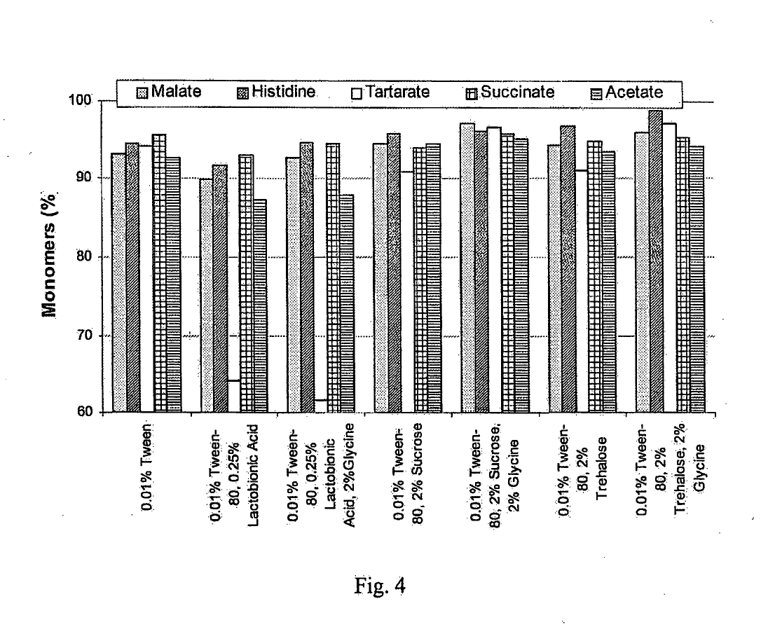

FIG. 4 shows the percentage monomer of cetuximab in various formulation conditions after incubation at 50° C for 72 hours. The percentage monomer was smallest in tartrate buffer and largest in histidine buffer. The formulation containing 25 mM histidine at pH 6.0 with 2% sugar (trehalose or sucrose) and with 0.25% lactobionic acid, 2% glycine and 0.01% Tween® had the highest monomer percentage.

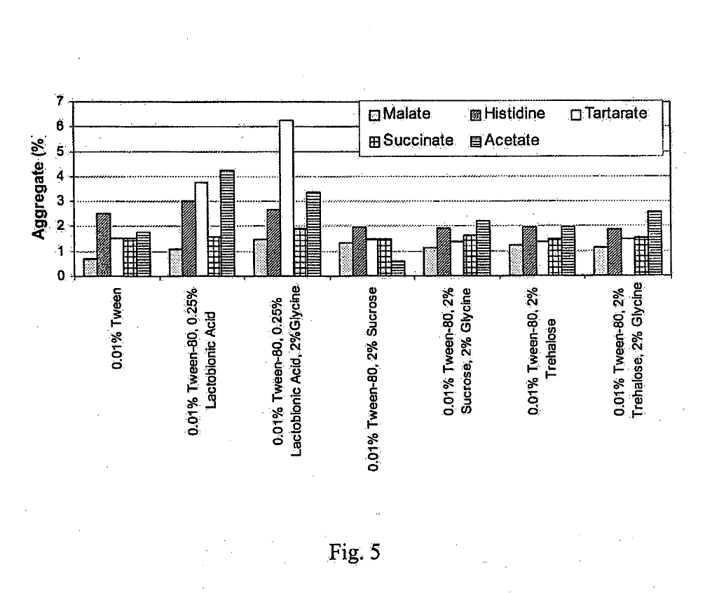

FIG. 5 shows the percentage of soluble aggregates of cetuximab in various formulation conditions after incubation at 50° C. for 72 hours. The percentage of soluble aggregates was largest in tartrate buffer and smallest in malate buffer.

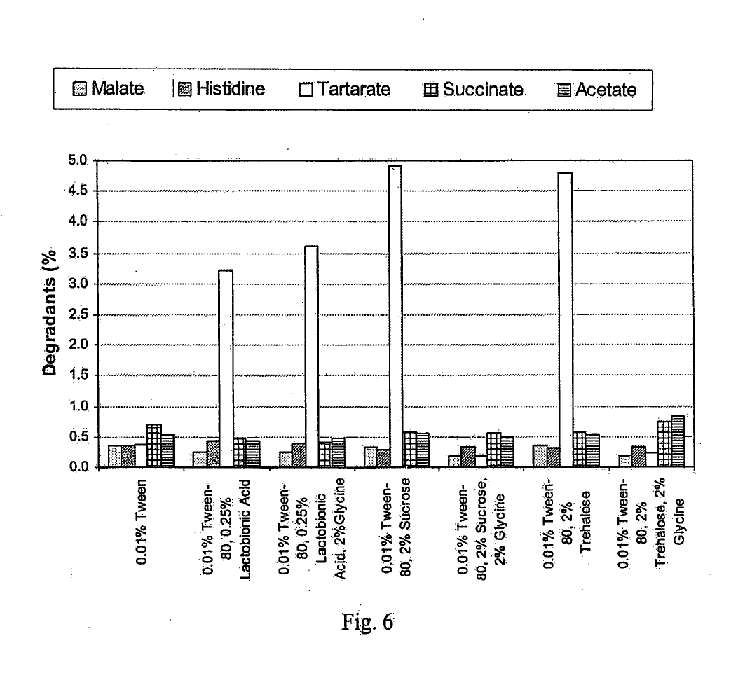

FIG. 6 shows the percentage of degradants of cetuximab in various formulation conditions after incubation at 50° C. for 72 hours. The percentage of degradants was less than 1% for all buffers, except in tartrate buffer where it was up to 5%.

### Example 3

### Lyophilization Process

Various solutions of cetuximab (50 mg/mL) in a histidine buffer (25 mM) at pH 6.0 were prepared by adding the following excipient(s):

(i) 2% glycine and 0.005% Tween 80®;

(ii) 2% trehalose and 0.005% Tween 80®;

(iii) 2% mannitol and 0.005% Tween 80®;

(iv) 1.875% glycine, 0.125% lactobionic acid, and 0.005% Tween 80®;

(v) 2% sucrose and 0.005% Tween 80®;

(vi) 1% glycine, 1% trehalose, and 0.005% Tween 80®;

(vii) 1% glycine, 1% sucrose, and 0.005% Tween 80®;

(viii) 1% glycine, 1% mannitol, and 0.005% Tween 80®;

One milliliter of each solution was lyophilized and then reconstituted with 1 mL milliQ water or less to achieve a final concentration of 50 mg/mL or more up to 200 mg/mL, For each sample, the reconstitution time was less than 1 minute, and the reconstituted solutions were particle free.

To test the long-term stability of lyophilized solutions, a sample of each lyophilized formulation was incubated at 60° C. for 4 days, and another sample was incubated at 60° C. for 11 days. The initial lyophilized sample was not incubated. The initial lyophilized sample and the incubated samples were reconstituted with 1 mL milliQ water. For all samples, the reconstitution time was less than a minute and the solutions were clear.

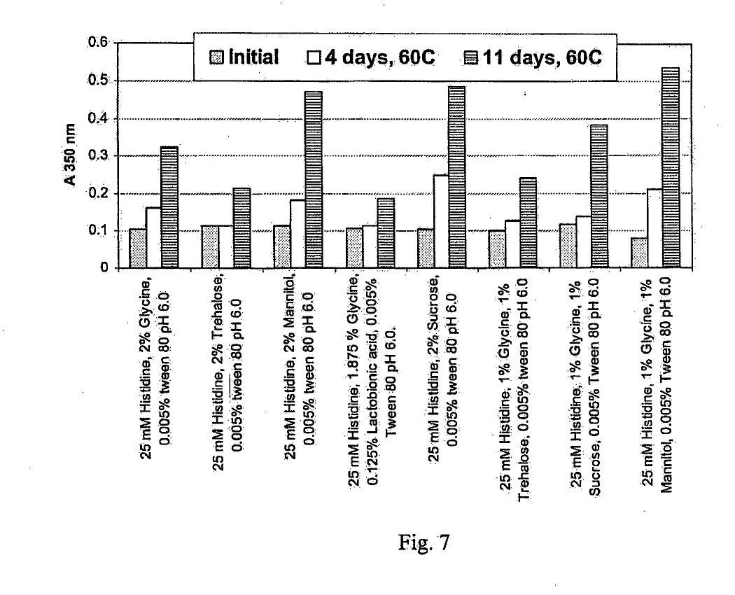

Turbidity was measured at 350 nm. FIG. 7 shows the turbidity of various reconstituted cetuximab lyophilized products at the initial stage, after 4 days of incubation at 60° C., and after 11 days of incubation at 60° C. The turbidities of all initial samples were comparable. The turbidities increased for all formulations with incubation time. The least solution turbidity change after reconstitution was in the formulation containing 25 mM histidine at pH 6.0 with 1.875% glycine, 0.125% lactobionic acid, and 0.005% Tween 80®.

SEC-HPLC analysis was used to measure monomer, aggregate and degradarit fraction content. The SEP-HPLC analysis suggested that each sample showed no insoluble aggregates:

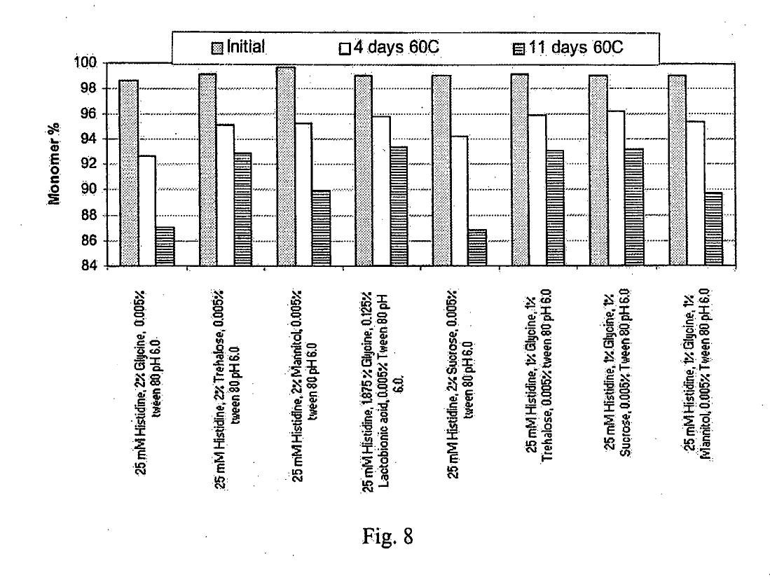

FIG. 8 shows the percentage monomer of the reconstituted cetuximab lyophilized products at the initial stage, after 4 days of incubation at 60° C., and after 11 days of incubation at 60° C. Initially, percentage monomers were similar for all formulations. Percentage monomers decreased in all formulations with incubation time: The least loss in percentage monomer was in the formulation containing 25 mM histidine, 2% glycine, and 0.005% Tween 80® at pH 6.0 and in the formulation containing 25 mM histidine, 2% sucrose, and 0.005% Tween 80® at pH 6.0.

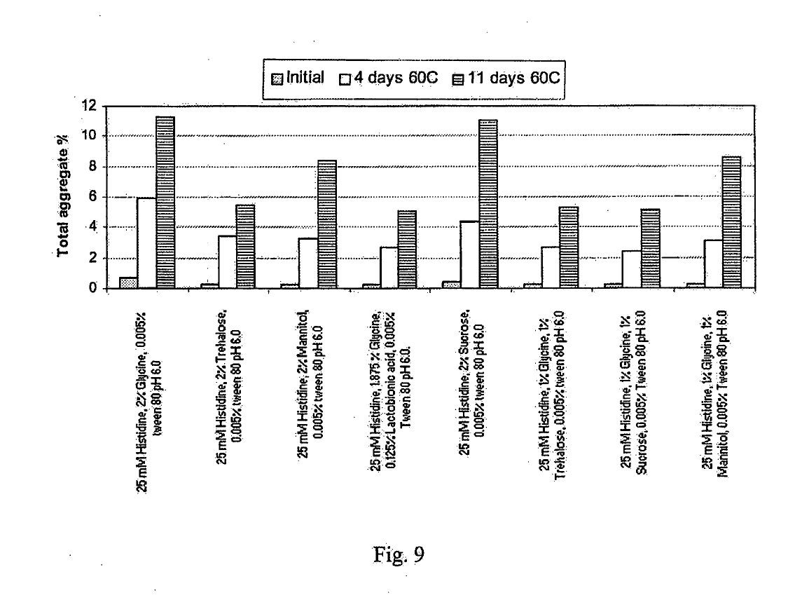

FIG. 9 shows the variation of percentage of soluble aggregates of the reconstituted cetuximab lyophilized products at the initial stage, after 4 days of incubation at 60° C., and after 11 days of incubation at 60° C. initially, percentage aggregrates were similar in all formulations. Percentage aggregates increased with incubation time in all formulations. Percentage aggregates were least both after 4 days and 11 days incubation time in the formulation containing 25 mM histidine, 1.875% glycine, 0.125% lactobionic acid, and 0.005% Tween 80® at pH 6.0. The largest percentage of aggregates was in the formulation containing 25 mM histidine, 2% glycine, and 0.005% Tween 80® at pH 6.0 and in the formulation containing 25 mM histdine, 2% sucrose, and 0.005% Tween 80® at pH 6.0.

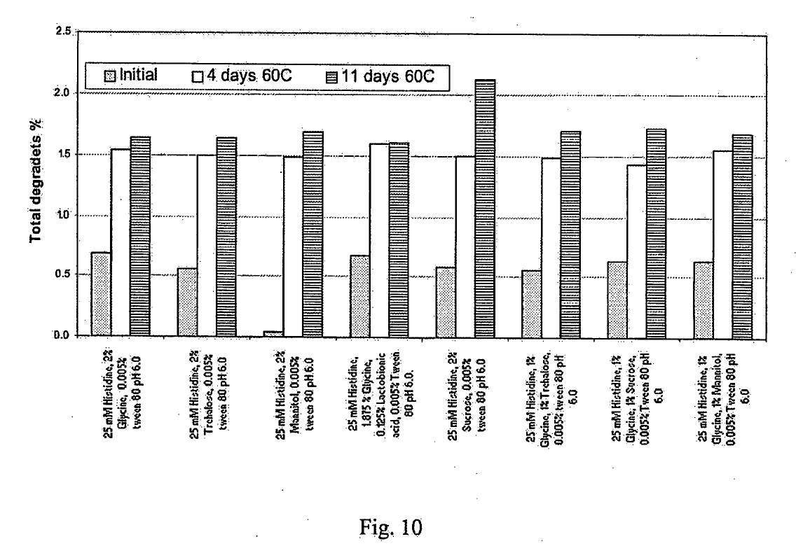

FIG. 10 shows the percentage of degradants of the reconstituted cetuximab lyophilized at the initial stage, after 4 days of incubation at 60° C., and after 11 days of incubation at 60° C. Before incubation, the percentage of degradants among the initial samples was least for the formulation containing 25 mM histidine, 2% mannitol, and 0.005% Tween 80®. Except for the formulation containing 25 mM histidine, 2% sucrose, and 0.005% Tween 80® at pH 6.0, all other formulation conditions have similar degradants after 4 and 11 days of incubation at 60° C. Percentage degradation was slightly larger for the formulation containing 25 mM histidine, 2% sucrose, and 0.005% Tween 80® at pH 6.0.

What is claimed is:

1. A lyophilized formulation comprising an anti-EGFR antibody, lactobionic acid and a buffer.

2. The lyophilized formulation of claim 1, wherein the anti-EGFR antibody is cetuximab.

3. The lyophilized formation of claim 1, wherein the buffer is histidine.

4. An aqueous formulation suitable for lyophilization, comprising an anti-EGFR antibody, lactobionic acid and a buffer.

5. The aqueous formulation of claim 4, wherein the anti-EGFR antibody is present at a concentration of from about 10 mg/mL to about 140 mg/mL.

6. The aqueous formulation of claim 5, wherein the anti-EGFR antibody is present at a concentration of from about 50 mg/mL to about 140 mg/mL.

7. The aqueous formulation of claim 5, wherein the anti-EGFR antibody is present at a concentration of about 50 mg/mL.

8. The aqueous formulation of claim 4, wherein the lactobionic acid is present at a concentration of from about 0.1% to about 0.5%.

9. The aqueous formulation of claim 8, wherein the lactobionic acid is present at a concentration of from about 0.125% to about 0.25%.

10. The aqueous formulation of claim 8, wherein the lactobionic acid is present at a concentration of about 0.125%.

11. The aqueous formulation of claim 4, wherein the buffer is selected from the group consisting of histidine, malate, tartrate, succinate, acetate and combinations thereof.

12. The aqueous formulation of claim 4, wherein the buffer has a pH of from about 5.5 to about 6.5.

13. The aqueous formulation of claim 12, wherein the buffer has a pH of about 6.0.

14. The aqueous formulation of claim 4, wherein the buffer is present at a concentration of from about 5 mM to about 50 mM.

15. The aqueous formulation of claim 14, wherein the buffer is present at a concentration of from about 10 mM to about 25 mM.

16. The aqueous formulation of claim 14, wherein the buffer is present at a concentration of about 25 mM.

17. The aqueous formulation of claim 4, further comprising a stabilizing agent selected from the group consisting of a polyhydric sugar, a sugar alcohol, a sugar derivative, an amino acid, a lyotropic salt and combinations thereof.

18. The aqueous formulation of claim 17, wherein the stabilizing agent is selected from the group consisting of mannitol, glycine and combinations thereof.

19. The aqueous formulation of claim 4, further comprising a surfactant.

20. The aqueous formulation of claim 19, wherein the surfactant is selected from the group consisting of polyoxyethylene(20)sorbitan monolaurate, polyoxyethylene(20)sorbitan monooleate, polyoxyethylene-polyoxypropylene block copolymer, bile salts and combinations thereof.

21. The aqueous formulation of claim 19, wherein the surfactant is polyoxyethylene(20)sorbitan monooleate.

22. The aqueous formulation of claim 19, wherein the surfactant is present at a concentration of from about 0.001% to about 0.01%.

23. The aqueous formulation of claim 22, wherein the surfactant is present at a concentration of from about 0.005% to about 0.01%.

24. The aqueous formulation of claim 22, wherein the surfactant is present at a concentration of about 0.005%.

25. An aqueous formulation of claim 4, wherein the anti-EGFR antibody is present at a concentration of about 50 mg/mL, the lactobionic acid is present at a concentration of about 0.125% and the histidine is present at a concentration of about 25 mM.

26. The aqueous formulation of claim 25, further comprising polyoxyethylene(20)sorbitan monooleate at a concentration of about 0.005%.

27. A lyophilized formulation made from the aqueous formulation of claim 4.

28. A method of stabilizing an antibody comprising lyophilizing an aqueous formulation comprising an anti-EGFR antibody, lactobionic acid, and a buffer.

29. The method of claim 28, wherein the buffer is histidine.

30. A method of treating a mammal, comprising administering a therapeutically effective amount of a reconstituted lyophilized formulation of claim 1, to a mammal, in need thereof.
